(19) United States
(12) Patent Application Publication
(10) Pub. No.: US 2021/0378552 A1
(43) Pub. Date: Dec. 9, 2021

(54) SMART MAT

(71) Applicants: Maurizio Macagno, Redmond, WA (US); Davide Vigano, Redmond, WA (US); Maria Pia Carmagnani, Redmond, WA (US); Stefano Rossotti, Redmond, WA (US)

(72) Inventors: Maurizio Macagno, Redmond, WA (US); Davide Vigano, Redmond, WA (US); Maria Pia Carmagnani, Redmond, WA (US); Stefano Rossotti, Redmond, WA (US)

(21) Appl. No.: 17/340,316

(22) Filed: Jun. 7, 2021

Related U.S. Application Data
(60) Provisional application No. 63/036,178, filed on Jun. 8, 2020.

(51) Int. Cl. A61B 5/11; A61B 5/00

(52) U.S. Cl. CPC A61B 5/1115; A61B 5/6894; A61B 5/742; A61B 5/1118

(57) ABSTRACT

The disclosure provides a system, apparatus, and method for monitoring pressure associated with a user sitting in a wheelchair for prolonged periods of time. By tracking the user's wheelchair pressure sitting history and analyzing the tracked data; relieving the user's sitting pressure and increasing blood circulation can be achieved through achieving higher levels of user adherence to performing pressure relief rehabilitation exercises.

### CROSS-REFERENCE TO RELATED APPLICATIONS

This application claims the benefit of priority under 35 U.S.C. § 120 to U.S. Provisional Patent Application Ser. No. 63/036,178, titled: “SMART MAT,” filed on Jun. 8, 2020, the entire disclosure of which is hereby incorporated by reference in its entirety for all purposes.

### TECHNICAL FIELD OF THE INVENTION

The present dis closure generally relates to sensor units and more specifically, to a sensor unit for sensing and characterizing forces placed by a user on a wheelchair.

### BACKGROUND OF THE INVENTION

Every year, around the world, between 250,000 and 500,000 people suffer a Spinal Cord Injury (SCI). The majority of spinal cord injuries are due to preventable causes such as road traffic crashes, falls or violence. People with a spinal cord injury are two to five times more likely to die prematurely than people without a spinal cord injury, with worse survival rates in low- and middle-income countries. Spinal cord injury is associated with lower rates of school enrollment and economic participation, and it carries substantial individual and societal costs. There is no reliable estimate of global prevalence, but estimated annual global incidence is 40 to 80 cases per million population. Up to 90% of these cases are due to traumatic causes, though the proportion of non-traumatic spinal cord injury appears to be growing. Pressure injuries are one of the most frequent secondary complications associated with wheelchair users with spinal cord injury. These injuries prevent regular wheelchair use, limiting independence and reducing quality of life. Pressure injuries are also associated with increased morbidity: complications arising from them are the second leading cause of death in individuals with SCI. Because of the serious consequences associated with pressure injuries, the preferred approach is prevention. Regular performance of pressure relief activities is a key component of prevention: these activities offload the skin and allow blood perfusion. However, adherence to pressure relief recommendations is low in individuals with SCI, in part due to the lack of sensation attributable to neuropathy.

In addition a large share of the aging population is wheelchair bound and needs to be monitored to improve health outcomes and reduce risk of pressure ulcer development. The older the person, the more delicate the skin and the higher the risk of pressure ulceration. Healing a pressure ulcer is a challenge in these cases. On average 60,000 people lose their life each year in the US due to complications directly related to pressure ulceration. According to top academic institutions and associations the cost of pressure ulceration in the US has skyrocketed in recent years to $11.6 billion dollars a year according to AHRQ (Agency for Healthcare Research and Quality).

The goal of improving health and reducing the risk of pressure ulcers remains paramount for the health of both spinal cord injured patients and the aged population using a wheelchair. Typically, users can avoid or minimize the occurrence of pressure ulcers by performing daily multiple recurring exercises that include leaning side to side, and performing this exercise on each side for 15 seconds every 15 minutes and/or 30 seconds every 30 minutes, for example. However, many wheelchair users tend to forget to perform these exercises, and often the standard protocol does not meet the needs and remove the risk for all wheelchair users and pressure ulcers remain a problem for all long term wheelchair users. Therefore, there is a need for monitoring wheelchair in-seat behavior that can characterize the movements of a particular user in order to avoid pressure ulcers, alert, and remind the user to perform the prescribed rehabilitation or prevention protocol for most wheelchair users with their own personalized regimen and a quantified approach.

### SUMMARY OF THE INVENTION

The disclosure addresses these needs and more by providing a system, apparatus, and a method for monitoring user's pressure associated with sitting in a wheelchair for prolonged periods of time. By tracking, analyzing, and relieving the pressure associated with using a wheelchair, and increasing blood circulation in the area of highest risk to make the user more adherent and also more comfortable and healthier.

In an embodiment, the disclosure provides a method of monitoring pressure associated with a user sitting in a wheelchair for prolonged periods of time, by tracking the user's wheelchair pressure sitting history; analyzing the tracked data; and relieving the user's sitting pressure through pressure relief rehabilitation exercises.

In other embodiments, the disclosure provides a method of monitoring pressure associated with a user sitting in a wheelchair for prolonged periods of time, by tracking the user's wheelchair pressure sitting history data using a plurality of textile pressure sensors present in a sitting mat or in a sensor infused cushion or in a cushion cover.

In other embodiments, the disclosure provides a method of monitoring pressure associated with a user sitting in a wheelchair for prolonged periods of time, by collecting the user's tracked pressure sitting history data through an electronic device; and transmitting the collected data to a mobile app on a smart phone.

In other embodiments, the disclosure provides a method of monitoring pressure associated with a user sitting in a wheelchair for prolonged periods of time, by transmitting the collected data wirelessly to the mobile app.

In other embodiments, the disclosure provides a method of monitoring pressure associated with a user sitting in a wheelchair for prolonged periods of time, by transmitting the collected data wirelessly from the mobile app to a cloud infrastructure or to a clinician's dashboard.

In other embodiments, the disclosure provides a method of monitoring pressure associated with a user sitting in a wheelchair for prolonged periods of time, by providing patient alerts and feedback to the patient for when it is time to move to relieve the pressure.

In other embodiments, the disclosure provides a method of monitoring pressure associated with a user sitting in a wheelchair for prolonged periods of time, by the sitting mat having one or more internal sensor boards for supporting the plurality of textile sensors; a detachable electronic device for collecting the tracked pressure history data; and a mating element or receptacle for receiving the plurality of textile sensors to the electronic device.

In other embodiments, the disclosure provides a method of monitoring pressure associated with a user sitting in a wheelchair for prolonged periods of time, by the one or more internal sensor boards having a double layer of fabric that acts as a substrate for instrumenting the plurality of textile sensors.

In other embodiments, the disclosure provides a method of monitoring pressure associated with a user sitting in a wheelchair for prolonged periods of time, by the one or more internal sensor boards having conductive traces that connect the plurality of textile sensors to form an electric circuit.

In other embodiments, the disclosure provides a method of monitoring pressure associated with a user sitting in a wheelchair for prolonged periods of time, wherein the pressure relief rehabilitation exercises include leaning side to side, leaning forward, or using the armrests to push up from the wheelchair.

### BRIEF DESCRIPTION OF THE DRAWINGS

Further characteristics of the invention will appear clearer from the detailed description that follows, referring to one of the purely exemplary embodiments and therefore, not limited as illustrated in the attached drawings, in which:

FIG. 1 illustrates an exemplary embodiment of the “smart mat” system for monitoring wheelchair use;

FIG. 2 illustrates another exemplary embodiment of the smart mat system for monitoring wheelchair use;

FIG. 3 illustrates an exemplary embodiment of the smart mat apparatus;

FIG. 4 illustrates an exemplary embodiment of the smart mat apparatus, which includes an internal sensor board;

FIG. 5 illustrates an embodiment of the smart mat system for remote patient monitoring;

FIG. 6A illustrates an embodiment of the mobile app on a smart phone;

FIG. 6B illustrates an embodiment of the mobile app on a smart phone;

FIG. 7A illustrates an embodiment of the mobile app screen relief exercise detection and feedback;

FIG. 7B illustrates another embodiment of the mobile app screen relief exercise detection and feedback;

FIG. 7C illustrates another embodiment of the mobile app screen relief exercise detection and feedback; and

FIG. 8 illustrates an embodiment of a clinician's dashboard, including a color-coded holistic view of patient population with drill down calendar view

### DETAILED DESCRIPTION OF THE EMBODIMENTS

The disclosure provides a system, apparatus, and a method for monitoring pressure associated with sitting in a wheelchair for prolonged periods of time. By tracking, analyzing, and relieving pressure associated with using a wheelchair, the user is made more comfortable and healthier.

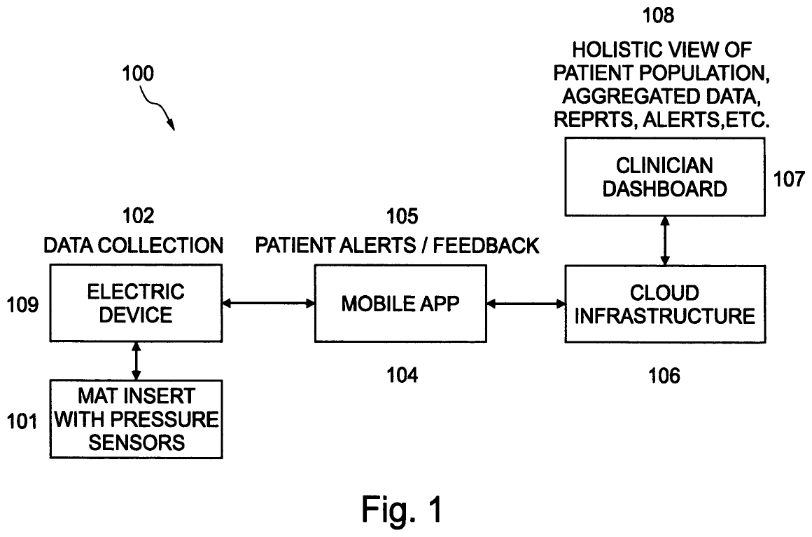

FIG. 1 illustrates an exemplary embodiment of the system, referred to herein as the “smart mat” system (100), for monitoring wheelchair use. In this example, a sitting cushion or sitting mat insert with a plurality of pressure sensors (101) can be used to generate and collect pressure data (102), which can be collected in an electronic device (103). The collected pressure data can be transmitted to a mobile app (104) to provide patient alerts and feedback (105) to the patient. The collected data can be further transmitted to a cloud infrastructure (106) and/or to a clinician's dashboard (107) to provide a holistic view of the patient population, aggregated data, reports and alerts etc. (108). In some embodiments, one or more Bluetooth devices, or other similar wireless devices, can be used to transmit the collected pressure data to the mobile app, cloud infrastructure, and/or the clinician's dashboard.

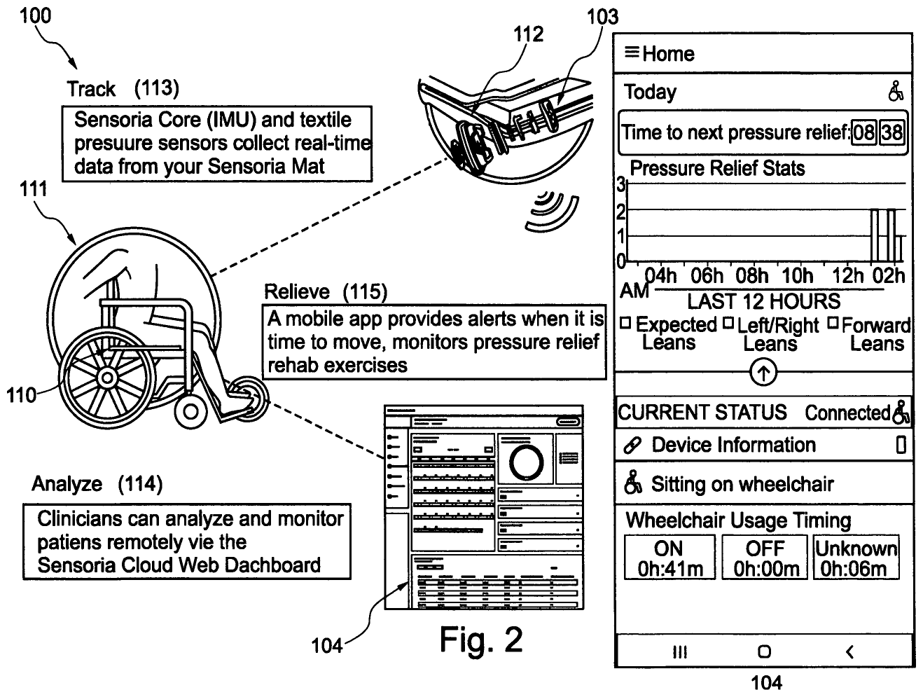

FIG. 2 illustrates another exemplary embodiment of the smart mat system (100). In this embodiment, the system includes a mat apparatus, referred to herein as a “smart mat” (110). In different embodiments, the smart mat includes a sensor infused cushion designed for wheelchair patients; a mobile app (104); and a clinician's dashboard (107).

The first step in monitoring pressure associated with sitting in a wheelchair is tracking the user's sitting history. As shown in this figure, when a user sits in a wheelchair (111) upon the smart mat (110), a plurality of textile pressure sensors are activated, and pressure data can be generated and collected by wiring to a receptacle (112) connected to a detachable electronic device (103). The pressure data can be generated, collected, and tracked in real time (113) and transmitted wirelessly to the mobile app (104) and/or to the clinicians dashboard (107) through one or more Bluetooth devices, or other similar wireless devices.

The next step in monitoring pressure associated with sitting in a wheelchair is analyzing the tracked user sitting history (114). In some examples, clinicians can analyze and monitor patients remotely using the cloud web-based dashboard (107), for example allowing them to follow up with wheelchair users that may need assistance in doing their relief exercises.

Finally, the last step in monitoring pressure associated with sitting in a wheelchair is relieving the pressure associated with sitting in a wheelchair for prolonged periods of time (115). In some examples, the mobile app (104) and/or the clinician's dashboard (107) through the mobile app can be used to provide alerts to the wheelchair user when it is time for the user to move and/or monitor pressure relief rehabilitation exercises. The system is also able to auto-detect the execution of a relief exercise (125), thus avoiding to alert the wheelchair user when not needed, and providing positive feedback on the execution and completion of the exercise.

Textile pressure sensors constitute an ideal choice as platforms for smart mat and smart cushion devices, since they are soft, flexible, lightweight, and can be used in many forms. This class of textiles with electronic capabilities has been referred to as electronic textiles (e-textiles). Depending on the type and application, e-textiles may be capable of sensing, data processing, actuation, and energy storage or generation. Textile based sensors provide an interface between the user and an electronic system by converting physiological or environmental signals into electrical signals. The smart mat is capable of monitoring variables such as strain, pressure, temperature, displacement, humidity, etc., and can be used in many applications including medical rehabilitation and health monitoring.

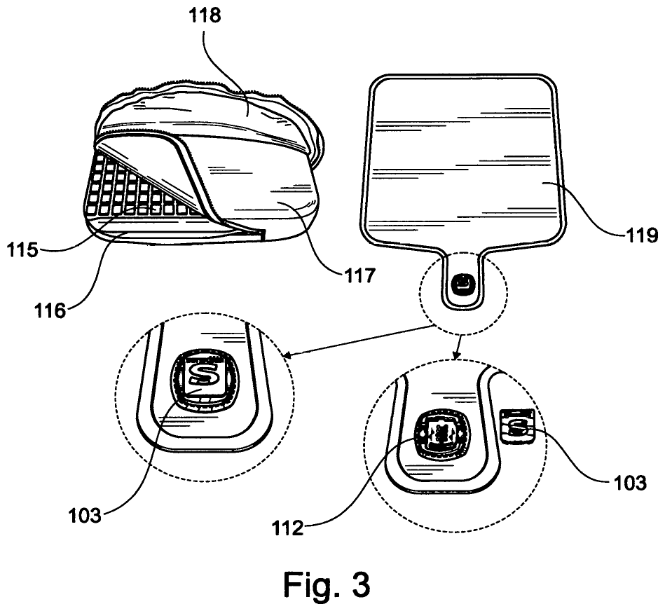

FIG. 3 illustrates an exemplary embodiment of the smart mat apparatus (110). While conventional wheelchair cushions are designed for comfort, pressure management, posture, and increased sitting stability, the smart mat apparatus is designed to maximize comfort by monitoring pressure, providing posture feedback, and monitoring rehabilitation and stability. The benefits of the smart mat include guiding daily rehabilitation routine, providing alerts and behavioral feedback, reducing risk of ulceration, and allowing sharing of data with a helper and clinician. As described herein, the smart mat apparatus features soft textile pressure sensors, Bluetooth (BT) device, a dedicated mobile app, and is rechargeable and washable.

As shown in this figure, the smart mat apparatus (110) includes cushion foam (115), a gel layer (116), a cushion inner cover (117), a cushion outer cover (118), a sensor board (119), a detachable electronic device (103), and a mating element or a receptacle (112) for the plurality of textile sensors. In some embodiments, the cushion foam provides climate control, pressure management, posture improvement, and increased sitting stability through gel and memory foam.

In some embodiments, the cushion cover (103/104) is removable. In other embodiments, the cushion cover is washable. In some embodiments, the electronics are rechargeable. In other embodiments, the soft textile presser sensors maximizes comfort, monitors presense, and reminds and monitors pressure relief activity.

In other embodiments, the sensor board (119), the detachable electronic device (103) and the mating element or receptacle (112) can be used with other type of commercially available cushions, providing a versatile system that can be applied on the preferred wheelchair user sitting cushion.

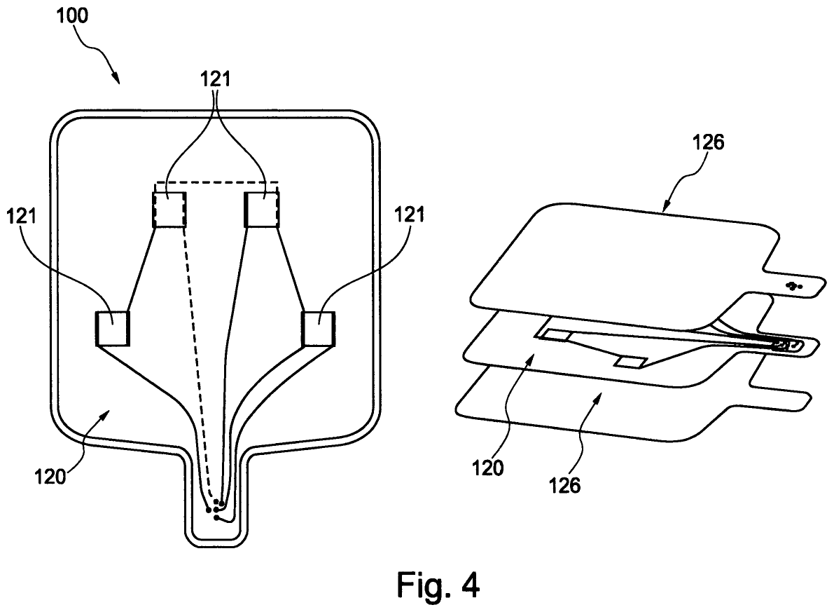

FIG. 4 illustrates an exemplary embodiment of the smart mat apparatus (110), which includes an internal sensor board (120). In some embodiments, the sensor board (120) includes a double layer of fabric that acts as a substrate for instrumenting textile sensors (121). The sensor board (120) can also include conductive traces that connect the textile sensors to form an electric circuit. In other embodiments, the sensor board includes components such as a stiff breathable fabric used as a substrate for the sensors. For example, Buckram fabric can be useful due to its stiffness and ease of pulling on and off the sensor board. Other components include, for example, 40 mm×40 mm textile sensors, conductive yarn traces, AFE female receptacle, and a core electronic device. In some embodiments, the sensor board (120) can be sandwiched in between an upper (126) and a bottom layer (128) of TPU, vinyl or other waterproof material, in order to preserve the sensor board (120) from damage due to water or other bodily fluids.

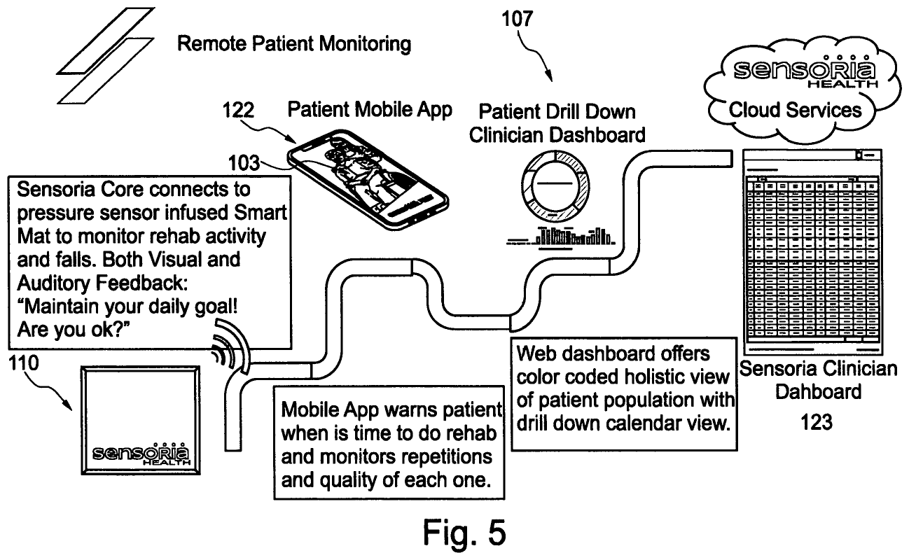

FIG. 5 illustrates an embodiment of the smart mat system for remote patient monitoring. In this figure, the electronic device connects to pressure sensor infused smart mat (110) to monitor rehabilitation activity and falls. The collected information is then sent wirelessly to the mobile app (104) on a smart phone (122). The mobile app provides both visual and auditory feedback, for example, “Maintain your daily goal! Are you ok?” The mobile app alerts the wheelchair user patient when it is time to do rehabilitation and monitors repetitions and quality of each one. Once the data is transmitted to clinician's dashboard (107), a web dashboard offers color coded holistic view of patient population with drill down calendar view (123).

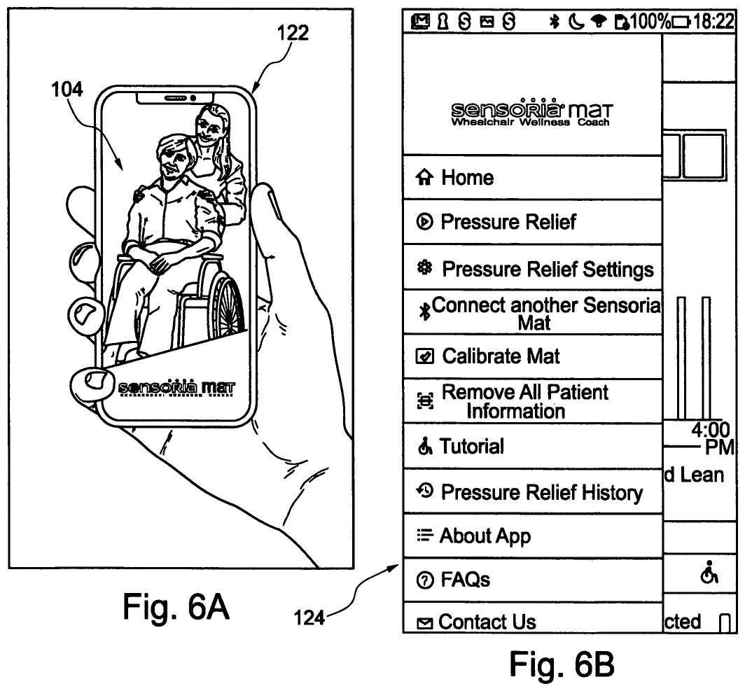

FIGS. 6A and 6B illustrate an embodiment of the mobile app (104) on a smart phone (122). When opened, the mobile app is generally showing a home screen (125) that display the current status of the system, including the time left to the next relief, and daily statistics. Optionally, the user can open a menu (124) of choices to select, including connecting to a mat system, visualizing pressure statistics, perform system calibration, remove all user data, and other accessory functions.

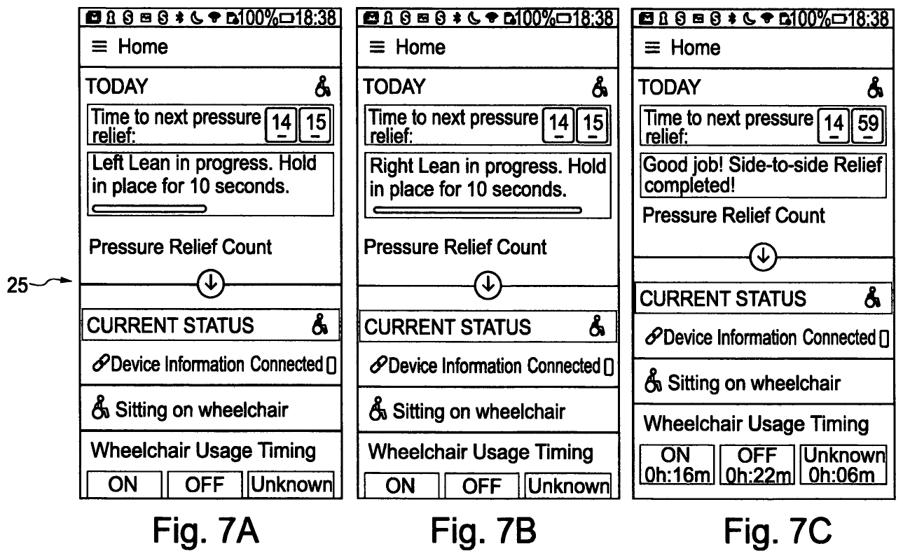

FIGS. 7A, 7B, and 7C illustrate an embodiment of the mobile app (104) screen display a relief in pressure progress. (125). In this case, the system has detected a side-to-side lean, and it's displaying in real time the exercise in progress and the time left to completion. At the end of a left lean, the user is invited to do a lean on the other side, and at the end of the two lean periods it informs the user of the completion of the exercise.

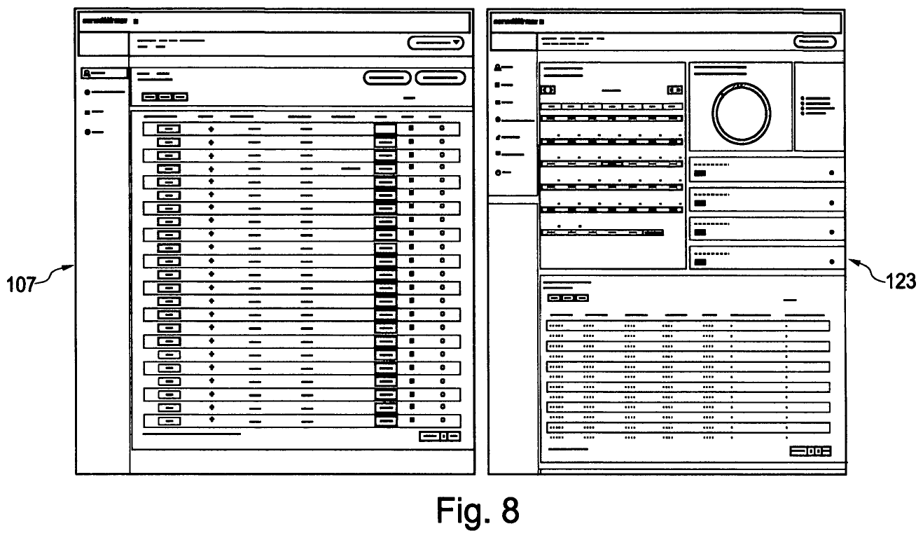

FIG. 8 illustrates an embodiment of a clinician's dashboard (107), including the color-coded holistic view of patient population with drill down calendar view (123).

While the inventive features have been particularly shown and described with reference to the disclosed embodiments, it will be understood by those in the art that the foregoing and other changes can be made therein without departing from the sprit and the scope of the disclosure. Likewise, the various diagrams may depict an example architectural or other configuration for the disclosure, which is done to aid in understanding the features and functionality that can be included in the disclosure. The disclosure is not restricted to the illustrated example architectures or configurations but can be implemented using a variety of alternative architectures and configurations. Additionally, although the disclosure is described above in terms of various exemplary embodiments and implementations, it should be understood that the various features and functionality described in one or more of the individual embodiments are not limited in their applicability to the particular embodiment with which they are described. They instead can be applied alone or in some combination, to one or more of the other embodiments of the disclosure, whether or not such embodiments are described, and whether or not such features are presented as being a part of a described embodiment. Thus, the breadth and scope of the disclosure should not be limited by any of the above-described exemplary embodiments.

1. A method of monitoring pressure associated with a user sitting in a wheelchair for prolonged periods of time, comprising:
tracking the user's wheelchair pressure sitting history;
analyzing the tracked data; and
relieving the user's sitting pressure through pressure relief rehabilitation exercises.

2. The method of claim 1, further comprising tracking the user's wheelchair pressure sitting history data using a plurality of textile sensors present in a sitting mat.

3. The method of claim 2, further comprising collecting the user's tracked pressure sitting history data in an electronic device; and transmitting the collected data to a mobile app on a smart phone.

4. The method of claim 3, further comprising transmitting the collected data wirelessly to the mobile app.

5. The method of claim 4, further comprising transmitting the collected data wirelessly from the mobile app to a cloud infrastructure or to a clinician's dashboard.

6. The method of claim 5, further comprising providing patient alerts and feedback to the patient for when it is time to move to relieve the pressure.

7. The method of claim 3, further comprising the sitting mat having one or more internal sensor boards for supporting the plurality of textile sensors; a detachable electronic device for collecting the tracked pressure history data; and a mating element or receptacle for receiving the plurality of textile sensors to the electronic device.

8. The method of claim 7, further comprising the one or more internal sensor boards having a double layer of fabric that acts as a substrate for instrumenting the plurality of textile sensors.

9. The method of claim 8, further comprising the one or more internal sensor boards having conductive traces that connect the plurality of textile sensors to form an electric circuit.

10. The method of claim 1, wherein the pressure relief rehabilitation exercises include leaning side to side.
